(19) United States
(12) Patent Application Publication
(10) Pub. No.: US 2007/0017516 A1
(43) Pub. Date: Jan. 25, 2007

(54) Device and process for providing gas mixtures

(76) Inventor: Klaus Schmidt, Gunzburg (DE)

Correspondence Address: HEAD, JOHNSON & KACHIGIAN, 228 W 17TH PLACE, TULSA, OK, 74119, US

(21) Appl. No.: 11/486,139

(22) Filed: Jul. 13, 2006

(30) Foreign Application Priority Data
Jul. 14, 2005 (DE) 102005032977.2-44

(51) Int. Cl. A61M 16/00

(52) U.S. Cl. 128/204.23

(57) ABSTRACT

An intubation hose, a supply regulator and a supply reservoir of a predetermined gas such as xenon, a gas mixing chamber connected to the supply regulator and the supply reservoir, and a selection element that separates the predetermined gas from other gases or separates other gases from the predetermined gas may be combined to form a respirator. The respirator delivers the predetermined gas supplied from the reservoir to a mixing chamber where the predetermined gas mixes with other gases that are then provided to a consumer such as a patient. Afterwards, the predetermined gas is at least partially recovered through the use of selection element. This recovery allows the recovered predetermined gas to be reused which substantially reduces the cost of operating the respirator.

### AREA OF APPLICATION

The invention regards the preparation of gas mixtures, in particular of breathing gases for respired patients

### TECHNICAL BACKGROUND

On demand provision of gas mixtures, wherein a predetermined concentration of single components is maintained, is state of the art.

When the complete gas mix is provided to a user not in a closed, but in an open cycle, and the target-fraction of the gas is partially consumed, and however the remainder of the target-fraction has to be reused or at least collected, this becomes much more complicated, especially depending on the quantities processed and depending on which target fraction relative to the other fractions is being dealt with.

When the target fraction is a noble gas like e.g. Xenon, the handling becomes rather more complicated.

In the medical field it has become apparent, that in particular an induction of Xenon into the breathing cycle of respired patients is helpful for sedating the patient and also for protecting the brain function of the patient.

The following facts, however make the use of this insight difficult in practice:
Xenon in the required quantities is rather expensive at a cost of US$ 15 per liter and a consumption of 6 liters per respiration minute per patient and
due to cost pressures the acquisition of new respiration equipment allowing the provision of Xenon is delayed

### DESCRIPTION OF THE INVENTION

### a) Technical Objective

Therefore the objective of the invention is to provide a device and a process, allowing the provision of an additional gas fraction, xenon in particular, within a gas mix, e.g. a breathing gas for a patient, in a very simple manner an with usage losses of xenon as small as possible.

### b) Solution

This objective is accomplished through the features of the claims 1, 15 and 16. Preferred embodiments can be derived from the dependent claims.

Through an adjustable supply regulator for the target fraction for preparing the breathing gas, the percentage of the target fraction, e.g. Xenon, in the breathing gas can be adjusted exactly according to requirements.

In case the remainder of the breathing gas did not yet contain xenon, since it was e.g. made from ambient air with the addition of oxygen, also no sensor for measuring the prior content of the target fraction is necessary.

If however the breathing return gas exhaled by the patient is to be recycled and reused as a breathing gas, if necessary through the addition of fresh oxygen, the percentage of the target fraction already contained in the return gas has to be measured through a sensor and has to be considered when adding the target fraction.

Independently from the above, in the path of the breathing return gas, between the intubation-hose and either the open outlet for the breathing return gas or the return into the gas mixing chamber for preparing the new breathing gas, a selection element is present for reusing the target fraction:

This can be an active separator, separating the target fraction form the rest of the gas mix, if the return gas shall be released into the atmosphere. This could be a centrifuge, or a simple membrane, which lets all other components of the return gas pass into the environment, but not the target fraction.

If the breathing return gas is recycled and used for producing fresh breathing gas, the target fraction included in the breathing return gas can be kept there, but for processing the breathing gas new and other fractions than the target fraction have to be added to (e.g. oxygen) or extracted from the breathing return gas (e.g. carbon dioxide).

Also here it has to be assured that when adding or subtracting other fractions, the target fraction is not subtracted unintentionally.

This can be performed through a passive selector e.g. a membrane, through which other fractions than the target fraction ca enter or exit the mixing chamber, but the target fraction cannot exit.

In this case, when recycling the breathing return gas for producing new breathing gas, the content of the target fraction in the breathing return gas or after processing the new breathing gas, but before adding fresh target fraction, has to be known and therefore has to be measured by a sensor.

The target supply connection is then controlled depending on the results of the sensor, preferably through an electric or electronic controller, which is preferably connected with the control system of the rest of the breathing apparatus, or even integrated into it.

If the target fraction is not actively separated from the rest of the breathing gas, a target exhaust connection for exhausting the mixture including the target fraction is provided in the part of the gas path, connected with the reservoir and/or the supply regulator for the target fraction, where preferably the sensor for the determination of the target fraction is located.

Thereby a separation of the target fraction form the remaining fraction is not necessary and only the required additional quantity of target fraction is inducted into the path of the gas mix, e.g. in a mixing chamber, which is therefore provided.

For this a bypass to the main gas path can be provided through the target supply and target exhaust connection, which is used for adding additional target fraction, being connected with the reservoir of the target fraction.

In case the remaining gas mix is not processed again, but released into the environment (open cycle of the main gas mix), on the side of the target supply and target exhaust opposite from the point of consumption a selection element, e.g. a selective membrane, which passes all fractions, besides the target fraction is necessary.

In case the main gas mix is recycled and reprocessed, such a selection element may not be necessary, e.g. when
no fractions have to be removed from the breathing return gas during the reprocessing and
fresh quantities added into the return gas are added e.g. through valves allowing passage into the gas cycle only in supply direction.

In order to provide a retrofitable ad on device for a conventional gas processing device like a respirator it is possible, in particular with recirculated breathing gas, and also with a closed cycle of the target fraction or of the gas mix including the target fraction, to provide sensor, supply regulator and also the supply connection for the reservoir of the target fraction connected with the supply regulator and a possibly not necessary pressure generator for the target fraction in a target controller as an integrated subassembly, which can be integrated into the gas path of the processor, e.g. forming a bypass conduit.

In case a selection unit is necessary, preferably also this selection unit, e.g. a selective membrane is located in the adapter, or in the target control unit.

In case of a y-shaped gas path this can be in the branch between the y-spot and the consumer, this means the patient, but also in the divided branches for exhalation and inhalation, wherein the target supply connection is located at least in the inhalation branch.

### c) Embodiments

Embodiments of the invention are subsequently described as examples in more detail. It is shown in:

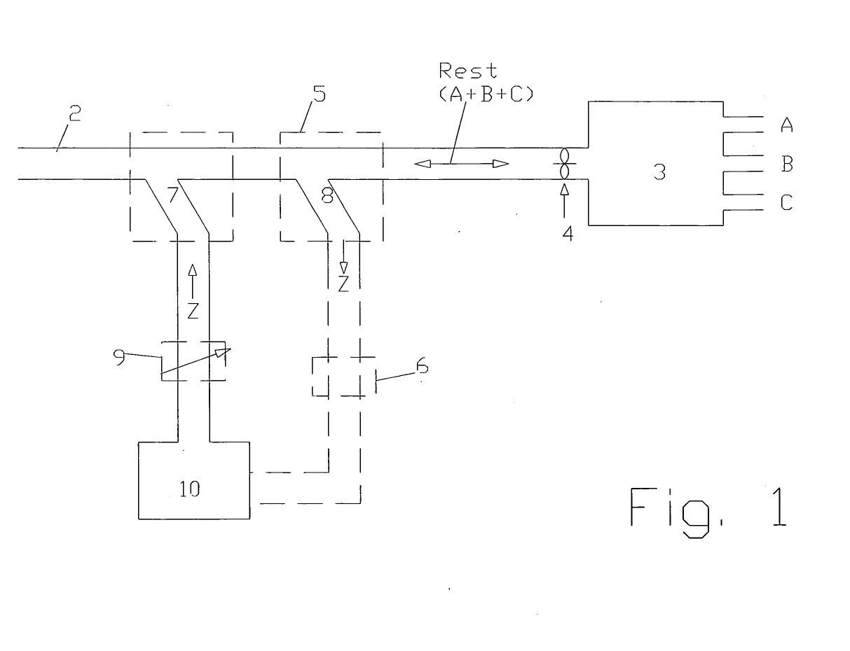

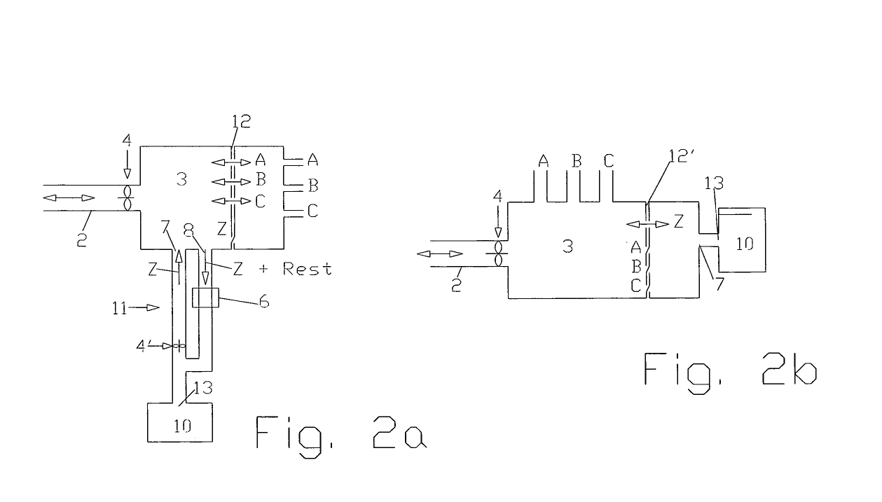

FIG. 1: a principle schematic of the process

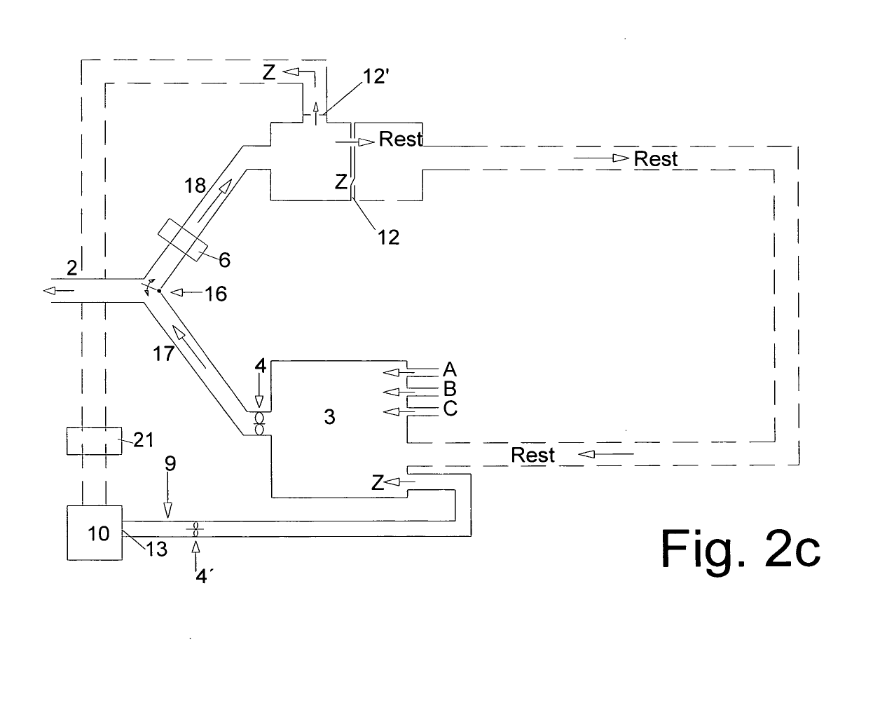

FIG. 2: several general ways to solve the problem and

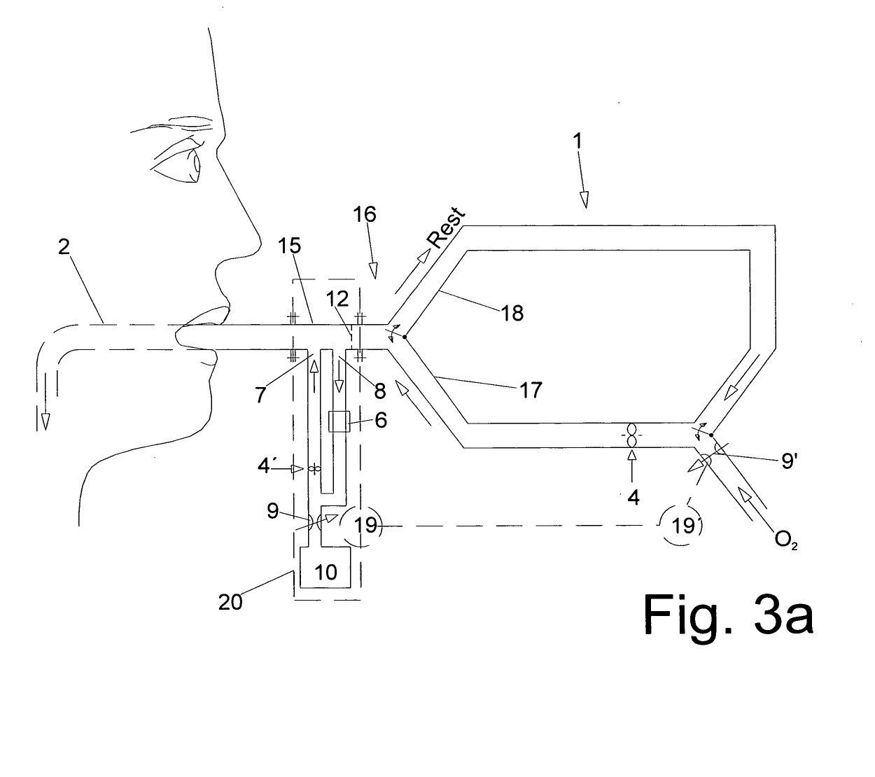

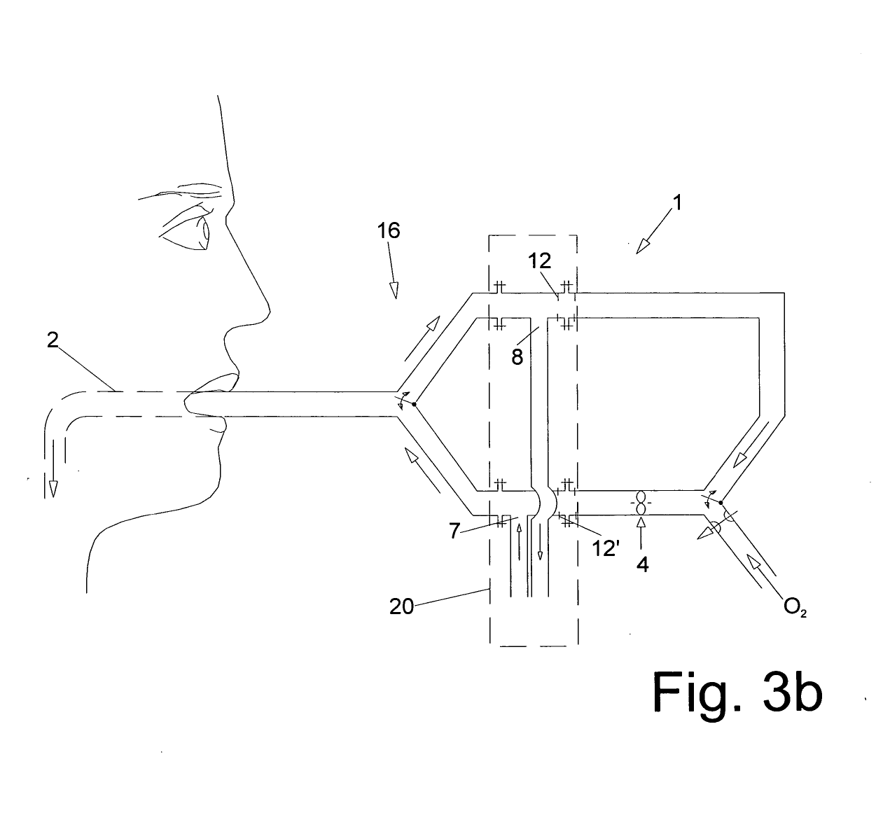

FIG. 3: detailed embodiments according to the invention.

FIG. 1 shows in principle, how in a mixing chamber (3) a gas mix is generated from several base components (A, B, C) and via a conduit, in this case a intubation hose (2), supplied to a consumer e.g. the patient. Therefore a pressure generator (4), e.g. a blower is integrated into the conduit. Into this conduit an additional target component (Z) shall be provided via the target supply connector (7), which is performed from the reservoir (10) via the supply connector (7) via a adjustable supply regulator (9).

The gas mix hereby is not only provided to the consumer through the conduit (2), but also the unused rest, this means the breathing return gas, is also returned again towards the mixing chamber (3).

In order not to loose the target fraction (Z) contained in the breathing return gas into the mixing chamber (3) and from there through the component supply lines to the outside, a selection element (5) is required in the path of the gas.

This can be an active separation device for separating the component (Z) from the gas mixture, whereby the target component (Z) can be returned into the reservoir or be reused otherwise. This can also only be a passive barrier, avoiding the reflux of the target component into the component inflows (A, B, C).

As shown in FIG. 2, for this purpose the target component (Z) can be provided, preferably directly into the mixing chamber, into the mixture out of the reservoir (10), preferably again through pressurization with a pressure generator (4′) between the reservoir (10) and the supply connection (7).

The content of target fraction in the gas mixture can be measured either in the mixing chamber (3) or in the supply line, e.g. the intubation hose (2) to the mixing chamber or also through extracting the complete mixture via a target exhaust connection (8) and supplying it again through a bypass to the supply connection (7) after replenishing additional target component (Z) from the reservoir (10), wherein before the supply the target fraction (Z) content is measured in the bypass through a sensor (6).

The supply connections for the additional components (A, B, C), which need to be partially added to the return gas in addition, also lead into the mixing chamber (3). An outflow of the target fraction (Z) through these supply connections (A, B, C) is avoided, either through them being located behind a membrane (12), which is not permeable for the component (Z), or by providing the components (A, B, C) in a manner, that they can only be passed in supply direction into the gas mixing chamber (3), no matter by which fraction.

FIG. 2b on the other hand shows a solution, where the mixing chamber has a membrane (12), which is only permeable for the target fraction (Z) and which is impermeable for the other components (A, B, C).

Accordingly the target supply connection (7), which is connected with the target feed connection (13) and in particular with the reservoir (10), is located in the mixing chamber on the opposite side from the outlet, this means on the side of the membrane (12′) opposing the intubation hose (2), while the connections (A, B, C) for the other components are located on the same side of the membrane as the outlet connection.

FIG. 2c shows a solution, where the inspiration path (17) and the expiration path (18) with their respective hoses run separate from the Y-piece (16), preferably with a flap for selectively opening one of the two hoses towards the central piece, the intubation hose.

In the embodiment in FIG. 2c, from the expiration hose (18) a separation of the target fraction (Z) is performed through a membrane (12′), while the remainder of the breathing return gas is either released into the environment, then however through a membrane (12) impermeable for the Z-fraction, or recirculated and provided to the gas mixing chamber (3) for preparing fresh breathing gas, whereby the membrane (12) is not necessary.

Into the mixing chamber for the fresh breathing gas additional target fraction (Z) is provided from a reservoir (10) though an adjustable supply regulator (9), whereby a pressure generator (4′) may be necessary in this supply line.

Into the reservoir (10) also the target fraction (Z) extracted from the expiration path (17) can be fed, wherein it's cleaning device (21) is located in this return path.

In this case the breathing gas or the breathing return gas is selectively run in an open or in a closed cycle, and also the separated target fraction (Z) is selectively run in a closed cycle, or recycled in spite of an open cycle.

The FIGS. 3 show a defined application of an applicator unit (20), which can be retrofitted on a conventional respirator (1), which respires a patient via a Y-piece (16) and via an intubation hose (2)

Thereby a closed cycle of the breathing gas or the breathing return gas is assumed, wherein for processing breathing gas from the return gas supplemental oxygen is added via an adjustable supply regulator (9) and,—what is not shown here—also CO₂ is extracted where necessary

According to FIG. 3a the applicator unit (20) is located between the intubation hose (2) and the Y-piece (16). The applicator unit (20) comprises a target adapter (15), which is located between the intubation hose (2) and the Y-piece (16) and from which a bypass (11) branches off analogous to the schematic in FIG. 2a, this means with a sensor (6) for measuring the content of the target fraction in the gas mix, a supply of target fraction through an adjustable supply regulator (9) from a target fraction reservoir (10) and if necessary with pressurization via a pressure generator (4′) located in the bypass conduit.

The supply regulator (9) is controlled by the control system (19), receiving signals form the sensor (6) and preferably connected with the control system (19′) of the respirator (1), or even integrated into it.

The membrane (12) drawn between the inlet- and outlet connections (7) and (8) for the target fraction (Z) and the Y-piece (16), which is permeable for all fractions besides the target fraction, prevents an inflow of the target fraction into the remaining cycle of the breathing gas. This is only necessary, in case the other inlets and outlets of this cycle would allow an outflow of the Z-component from the cycle. If this is prevented through special inlet valves, e.g. for the oxygen, the membrane (12) can be omitted.

FIG. 3b shows a solution, wherein the target supply connection (7) and the target outflow connection (8) for the target component (Z) are located on the side of the Y-connection (16) pointing away from the patient, in the expiration conduit (18) of the target outlet connection (8) and in the inspiration conduit (17) of the target supply connection (7), when the target connection (Z) together with the rest is also here run in the bypass conduit (11) in a closed loop.

Also here in each of them a membrane (12) or (12′) is required, in case the rest of the cycle of the breathing gas, e.g. the in feed for the oxygen does not prevent the outflow of the target fraction by design. If this is the case, the membranes (12), (12′) can also be omitted here.

Then it can also be possible to leave the target outlet connection (8) out completely, if the sensor (6) is instead located directly in the cycle of the breathing gas, preferably directly in front of the target supply connection (7).

The target supply connection (7) is then connected with the reservoir (10) only via the adjustable supply regulator (9).

### REFERENCE NUMERALS

1 Respirator
2 Intubation tube
3 Gas miixing chamber
4, 4′ Pressure generator
5 Selection element
6 Sensor
7 Target supply connection
8 Target outlet cnnection
9 Supply regulator
10 Reservoir
11 Bypass
11a, b Bypass conduits
12, 12′ Membrane
13 Target feed connection
14 Target control system
15 Target adapter
16 Y-piece
17 Inspiration hose
18 Exspiration hose
19 Controller
20 Applicator unit
21 Purging device

What is claimed is:

1. A respirator comprising:
an intubation hose;
a gas mixing chamber;
a supply connection connected to the gas mixing chamber and connected with a supply regulator and a supply reservoir of a predetermined gas; and
a selection element that separates the predetermined gas from other gases or separates other gases from the predetermined gas,
wherein either
the selection element is between the intubation hose and the mixing chamber, or
the mixing chamber is between the intubation hose and the selection element.

2. The respirator according to claim 1 further including at least one sensor that measures a concentration of the predetermined gas is provided in a gas path of the respirator, and wherein the at least one sensor controls the supply regulator.

3. The respirator according to claim 1 wherein the selection element is an active separation device.

4. The respirator according to claim 1 wherein the selection element is a passive selective barrier with a different permeability for the predetermined gas and the other gases.

5. The respirator according to claim 4 wherein the different permeability for the predetermined gas and the other gases depends on the direction of passage through the passive selective barrier.

6. The respirator according to claim 4 wherein the passive selective barrier is a membrane.

7. The respirator according to claim 1 wherein a pressure generator is connected with the supply connection.

8. The respirator according to claim 1 wherein an outlet connection is provided in a section of a gas path connected with either or both of the reservoir and the supply regulator.

9. The respirator according to claim 8 further including at least one sensor that measures a concentration of the predetermined gas is provided in a gas path of the respirator, and a bypass connected with the supply reservoir that does not pass through the selection element.

10. The respirator according to claim 1 wherein the selection element is a membrane that is permeable in both flow directions to the predetermined gas but is impermeable in both directions to the other gases, wherein an outlet connection for the predetermined gas is located on a side of the membrane with only the predetermined gas.

11. The respirator according to claim 1 wherein the selection element is a membrane that is permeable in both flow directions to the other gases but is impermeable in both directions to the predetermined gas, and wherein the supply connection and an outlet connection originate on a side of the membrane with the supply connection.

12. The respirator according to claim 1 further including at least one sensor that measures a concentration of the predetermined gas, and wherein the at least one sensor, the supply regulator and a feed connection for the supply reservoir are located in a control unit as an integrated unit that is connectable via the bypass conduits with the respirator.

13. The respirator according to claim 12 wherein the supply reservoir is located in the control unit as part of the integrated unit.

14. The respirator according to claim 13 wherein the pressure generator is located in the control unit as part of the integrated unit.

15. The respirator according to claim 1 further including an adapter in a gas path for connecting a control unit, the control unit having the selection element, and wherein the adapter can be inserted as a connector between the intubation hose and the gas mixing chamber.

16. The respirator according to claim 15 wherein the adapter is between the intubation hose and a Y-piece.

17. The respirator according to claim 15 wherein the selection element is a membrane.

18. The respirator according to claim 1 further comprising:
an adapter;
bypass conduits;
a control unit; and
wherein the adapter, the bypass conduits and the control unit form a uniform and retrofitable applicator-unit.

19. The respirator according to claim 1 wherein the supply regulator is coupled with a control system, the supply regulator and the control system are jointly able to maintain a constant concentration of the predetermined gas.

20. The respirator according to claim 1 wherein the supply regulator is coupled with an electric or electronic control system and at least one sensor,
wherein the supply regulator, the electric or electronic control system and the at least one sensor are jointly able to maintain a constant concentration of the predetermined gas.

21. The respirator according to claim 1 wherein the predetermined gas is a noble gas.

22. The respirator according to claim 21 wherein the noble gas is xenon.

23. The respirator according to claim 1 wherein a control system for the predetermined gas is integrated into a control system of the respirator.

24. A process for recovering and mixing a predetermined gas in a gas mixture comprising:
providing a gas mixture in a closed loop;
partially consuming at least a predetermined gas by a consumer;
measuring a concentration of the predetermined gas in the gas mixture at least at one location in the closed loop;
comparing the concentration of the predetermined gas with a selected concentration of the predetermined gas; and
controlling a supply of the predetermined gas based upon the comparing the concentration of the predetermined gas.

25. A process according to claim 24 wherein measuring a concentration of the predetermined gas is measured continuously.

26. A process according to claim 24 wherein measuring a concentration of the predetermined gas is performed directly after the consumer.

27. A process according to claim 26 further including supplying the gas mixture with an additional amount of the predetermined gas, and wherein the measuring the concentration of the predetermined gas is performed in a bypass that extracts the gas mixture.

28. A process according to claim 24 further including supplying the gas mixture with an additional amount of the predetermined gas, and wherein the measuring the concentration of the predetermined gas is performed in a bypass that extracts the gas mixture.

29. A process for reusing and additively mixing a predetermined gas in a gas mixture comprising:
providing a gas mixture in an open cycle; and
separating a predetermined gas from the gas mixture before an end of the open cycle which is then supplied to a reservoir of the predetermined gas after a time delay.

30. A process according to claim 29 wherein the time delay is for recycling and compressing the predetermined gas.
